(19) United States
(12) Patent Application Publication
(10) Pub. No.: US 2015/0342458 A1
(43) Pub. Date: Dec. 3, 2015

(54) OPHTHALMIC APPARATUS

(71) Applicant: CANON KABUSHIKI KAISHA, Tokyo (JP)

(72) Inventors: Kazuhiro Watanabe, Tokyo (JP); Sakiko Yamaguchi, Tokyo (JP); Naofumi Sekine, Kawasaki-shi (JP); Tetsuya Sekine, Kawasaki-shi (JP); Hideki Hayashi, Kiyose-shi (JP); Wataru Kaku, Yokohama-shi (JP)

(21) Appl. No.: 14/722,693

(22) Filed: May 27, 2015

(30) Foreign Application Priority Data
May 30, 2014 (JP) 2014-112662

(51) Int. Cl. A61B 3/12; A61B 3/14; G02B 27/64; A61B 3/00

(52) U.S. Cl. CPC A61B 3/12; A61B 3/0041; A61B 3/0083; A61B 3/14; G02B 27/646

(57) ABSTRACT

An ophthalmic apparatus includes an image-capturing unit that captures an image of an eye to be examined, a main body having in a rod-like shape and that accommodates the image-capturing unit, an eyepiece portion that is positioned at a first end of the main body in an axial direction of the main body and that is brought into contact with a portion around the eye to be examined, and a display that is positioned at a second end of the main body in the axial direction of the main body and that displays an image of the eye to be examined. The main body includes a holding portion that is to be held by a hand of an examiner and that extends in the axial direction of the main body.

### BACKGROUND OF THE INVENTION

1. Field of the Invention

The present invention relates to an ophthalmic apparatus that captures an image of an eye to be examined.

2. Description of the Related Art

A stationary ophthalmic apparatus and a hand-held ophthalmic apparatus are known examples of ophthalmic apparatuses that capture images of eyes to be examined. In order to capture an image of an eye to be examined, specifically, an eye fundus, it is necessary to stably maintain the position and angle of the ophthalmic apparatus with respect to the face of a patient during a period when image capturing is being performed by bringing a portion of the ophthalmic apparatus into contact with the face of the patient.

In general, in the case where a stationary ophthalmic apparatus is used, the position and angle of the stationary ophthalmic apparatus with respect to the face of a patient are stably maintained by supporting the stationary ophthalmic apparatus with respect to the face of the patient at two or more points by using a forehead rest with which the forehead of the patient is to be in contact, a chin rest on which the chin of the patient is to be placed, and the like.

On the other hand, in the case where a hand-held ophthalmic apparatus is used, since the ophthalmic apparatus is to be hand-held by an examiner, providing a chin rest or the like is difficult. Thus, the position and angle of the hand-held ophthalmic apparatus with respect to the face of a patient are generally stably maintained by pressing a forehead rest against the forehead of the patient or by pressing an eyepiece portion, which is brought close to an eye to be examined, against the face of the patient.

Japanese Patent Laid-Open No. 8-164114 discloses a hand-held ophthalmic apparatus that includes a hand-held portion, which is to be held by an examiner, and a forehead rest, which is brought into contact with the forehead of a patient.

Japanese Patent Laid-Open No. 9-285446 discloses a hand-held ophthalmic apparatus that includes a measuring component that measures an eye to be examined, an image-capturing unit that captures an image of the eye to be examined, and a display that displays an image of the eye to be examined captured by the image-capturing unit.

Japanese Patent Laid-Open No. 62-122640 discloses a built-in, portable corneal-thickness-ultrasonic-measuring instrument that includes an elongated body having a shape similar to a pen, which can easily be held in a hand. The elongated body includes a housing, which has a recess portion on the distal side thereof so as to be easily held in a hand, and a digital display, which is disposed on a surface of a top portion of the elongated body.

In each of the cases where ophthalmic apparatuses of the related art including the ophthalmic apparatuses disclosed in the above-mentioned patent documents are used, it is difficult for an examiner to stably maintain the position and angle of the ophthalmic apparatus with respect to the face of a patient by hand-holding the ophthalmic apparatus. In the ophthalmic apparatus disclosed in Japanese Patent Laid-Open No. 8-164114 and the ophthalmic apparatus disclosed in Japanese Patent Laid-Open No. 9-285446, a hand-held portion, which is held by an examiner, is connected to an apparatus body, which is brought into contact with the face of a patient, in such a manner that the hand-held portion projects substantially perpendicularly from the apparatus body. In the case where an ophthalmic apparatus includes such a hand-held portion, which is a so-called pistol grip, contact between the apparatus body and the face of the patient is adjusted by changing the way in which the examiner extends their arm, and on the other hand, the angle at which the apparatus body is in contact with the face of the patient is adjusted by the examiner changing the angle of their wrist (see FIG. 6A). However, when a force in a direction (eye axis direction) from the face of the patient toward the back of the patient's head is applied to the apparatus in a state where the apparatus is in contact with the face of the patient, a force also acts in a direction in which the apparatus rotates, and thus, it is difficult to stably maintain the position and angle of the apparatus. Consequently, there has been a problem in that deviations are likely to occur in the position and angle of the apparatus when the apparatus is in contact with the face of the patient. In order to address such a problem, in a workplace where a pistol-grip ophthalmic apparatus is used, an examiner maintains the position and angle of the ophthalmic apparatus with respect to the face of a patient by placing their unoccupied hand on an eyepiece portion (see FIG. 6B).

A measuring component included in the ophthalmic apparatus disclosed in Japanese Patent Laid-Open No. 62-122640 does not optically measure an eye to be examined, and there is less need to stably maintain the position and angle of the ophthalmic apparatus with respect to the face of a patient compared with an ophthalmic apparatus that performs optical measurement. Accordingly, the ophthalmic apparatus disclosed in Japanese Patent Laid-Open No. 62-122640 does not include an eyepiece portion that is brought close to an eye to be examined.

### SUMMARY OF THE INVENTION

The present invention is directed to an ophthalmic apparatus with which its position and angle with respect to the face of a patient can be stably maintained when hand-held by an examiner capturing an image of an eye to be examined.

An ophthalmic apparatus according to an aspect of the present invention includes an image-capturing unit that captures an image of an eye to be examined, a main body having a rod-like shape and that accommodates the image-capturing unit, an eyepiece portion that is positioned at a first end of the main body in an axial direction of the main body and that is brought into contact with a portion around the eye to be examined, and a display that is positioned at a second end of the main body in the axial direction of the main body and that displays an image of the eye to be examined. The main body includes a holding portion that is to be held by a hand of an examiner and that extends in the axial direction of the main body.

Further features of the present invention will become apparent from the following description of exemplary embodiments with reference to the attached drawings.

### BRIEF DESCRIPTION OF THE DRAWINGS

FIG. 1 is a diagram illustrating a schematic configuration of an ophthalmic apparatus according to an embodiment.

FIG. 2 is a flowchart illustrating a process that is performed by the ophthalmic apparatus according to the embodiment.

FIG. 3 is a perspective view illustrating the appearance of the ophthalmic apparatus according to the embodiment.

FIG. 4 is a diagram illustrating an exemplary usage of the ophthalmic apparatus according to the embodiment.

FIGS. 5A and 5B are perspective views illustrating a display of the ophthalmic apparatus according to the embodiment as seen from the side opposite to a display surface.

FIGS. 6A and 6B are diagrams illustrating an exemplary usage of a pistol-grip ophthalmic apparatus.

### DESCRIPTION OF THE EMBODIMENTS

An embodiment of the present invention will be described below with reference to the drawings.

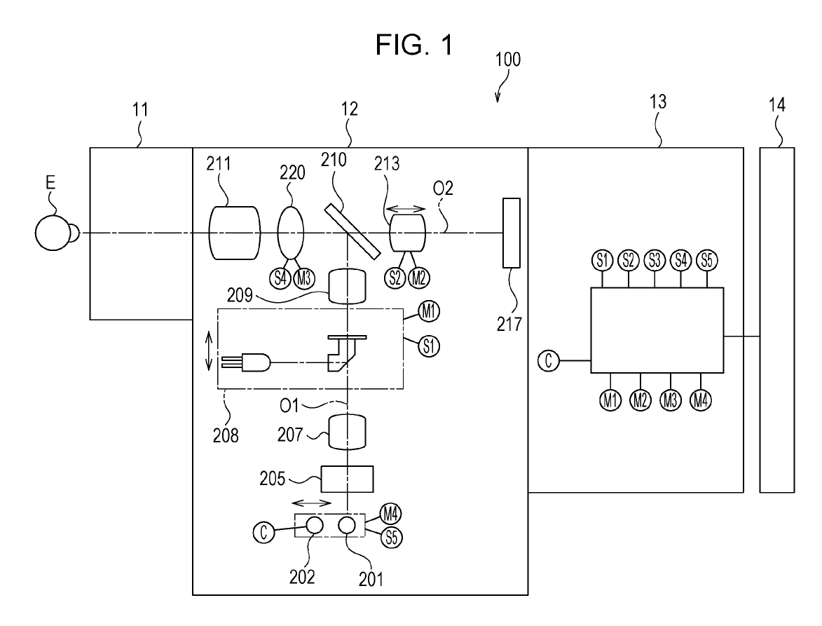

FIG. 1 is a diagram illustrating a schematic configuration of an ophthalmic apparatus 100 according to the embodiment.

The ophthalmic apparatus 100 includes an eyepiece portion 11, an image-capturing unit 12, a control unit 13, and a display 14.

The eyepiece portion 11 is brought into contact with the face of a patient such that the distance between the image-capturing unit 12 and a to-be-examined eye E is kept constant. The eyepiece portion 11 can be made of a flexible material. In this case, when the eyepiece portion 11 is brought into contact with the face of the patient, the position and angle of the ophthalmic apparatus 100 are likely to be stable, and in addition, the amount of pain experienced by the patient as a result of bringing the eyepiece portion 11 into contact with their face is reduced. Alternatively, the eyepiece portion 11 can have a gap therein or can be made of a transparent material. In this case, an examiner can bring the eyepiece portion 11 into contact with the face of a patient while visually examining an eye to be examined, and thus, deviation in the positions of the ophthalmic apparatus 100 and the eye to be examined can be prevented from occurring or the probability of such deviation occurring can be reduced. Alternatively, the eyepiece portion 11 can be made of a light-shielding material. In this case, an eye to be examined can be shielded from external light.

The image-capturing unit 12 includes an infrared LED light source 201, a white LED light source 202, a capacitor C, an illuminating-light path O1, an image-capturing optical axis O2, a ring slit 205, an illumination relay lens 207, an illumination relay lens 209, a split unit 208, and a perforated mirror 210. In addition, the image-capturing unit 12 includes an objective lens 211, a focus lens 213, an imaging device 217, and an anterior-eye-observation lens 220.

The infrared LED light source 201 is used to observe an eye fundus of a patient under infrared light. The white LED light source 202 is used to capture an image of an eye fundus of a patient under white light. The infrared LED light source 201 and the white LED light source 202 are placed in and removed from the illuminating-light path O1 by a driving motor M4. A position sensor S5 senses the positions of the infrared LED light source 201 and the white LED light source 202.

The ring slit 205 is a mask used for causing illuminating light emitted by the infrared LED light source 201 or the white LED light source 202 to become ring-shaped light. The ring-shaped light is focused onto the to-be-examined eye E by the illumination relay lens 207 and the illumination relay lens 209.

The capacitor C supplies power to the white LED light source 202. The capacitance of the capacitor C differs according to an image-capturing mode, and the capacitor C is appropriately charged and discharged each time the image-capturing mode is changed. Charging and discharging of the capacitor C is controlled by the control unit 13.

The split unit 208 includes a light source used for projecting a focus target, a moving mechanism that enters, when the to-be-examined eye E is observed, the illuminating-light path O1 and moves in the direction of an arrow in FIG. 1 in such a manner as to cause the focus target to move in an optical axis direction, and an advance and retreat mechanism that causes the focus target to retreat from the illuminating-light path O1 when image capturing is performed. A split-drive motor M1 drives the split unit 208 so as to move in the direction of the arrow in FIG. 1 to adjust the focal position of the focus target. A split position sensor S1 detects a position of the split unit 208, such as a stop position of the split unit 208.

The perforated mirror 210 is a total reflection mirror with a hole formed at the center thereof. The perforated mirror 210 reflects the ring-shaped light by using an outer peripheral mirror thereof and allows image-capturing light to pass through the center hole of the perforated mirror 210. The ring-shaped light that has passed through the perforated mirror 210 is caused to be focused onto the to-be-examined eye E by the objective lens 211 to illuminate the to-be-examined eye E. Light that has been reflected by the to-be-examined eye E passes through the objective lens 211 and is focused at the center of the perforated mirror 210. The image-capturing optical axis O2 is an optical axis extending from the to-be-examined eye E of the patient to the imaging device 217.

When the anterior-eye-observation lens 220 is placed in the image-capturing optical axis O2, an anterior eye can be observed. On the other hand, when the anterior-eye-observation lens 220 is not placed in the image-capturing optical axis O2, an eye fundus can be observed. Placement and removal of the anterior-eye-observation lens 220 in line with and from the image-capturing optical axis O2 are performed by using an anterior-eye-observation-lens drive motor M3 and an anterior-eye-observation-lens position sensor S4. The control unit 13 performs switching control of placement and removal of the anterior-eye-observation lens 220.

The focus lens 213 is a lens that is used for performing focus adjustment of the image-capturing light flux, which has passed through the center hole of the perforated mirror 210, and performs focus adjustment of the image-capturing light flux by moving in the direction of the other arrow in FIG. 1. A focus-lens drive motor M2 drives the focus lens 213 in response to a pulse from the control unit 13. A position sensor S2 detects the position of the focus lens 213.

The imaging device 217 converts the image-capturing light into an electrical signal by photoelectric conversion. The electrical signal obtained as a result of the photoelectric conversion is converted into digital data by the control unit 13. When an observation using infrared light is performed, an observation image is displayed on the display 14, and when image capturing is performed, image data is recorded to a memory or a storage medium (not illustrated). Digital data may be transmitted via a transmission unit or the like.

The control unit 13 performs drive control of the drive motors M1 to M4, control of the sensors S1 to S5, control in response to an input from a user interface, control of image data processing, control of displaying a captured image on the display 14, and the like.

The control unit 13 includes a processor and a memory. As a result of the processor of the control unit 13 executing a program stored in the memory, various drive controls, sensing control, data processing, data input/output, and display control are realized.

For example, a liquid crystal display can be used as the display 14. The display 14 displays an image of a to-be-examined eye that is undergoing image capturing, an image of a to-be-examined eye stored in the memory, and the like in response to display control by the control unit 13. Note that the display 14 may display an image of a to-be-examined eye while superposing, on the image of the to-be-examined eye, data items such as the name, age, and sex of a patient and a data item indicating whether the to-be-examined eye is the right eye or the left eye of the patient or may simply display the image of the to-be-examined eye. A touch panel may be integrally disposed with a display surface of the display 14 in such a manner that the display 14 serves as a user interface through which an examiner performs an input operation.

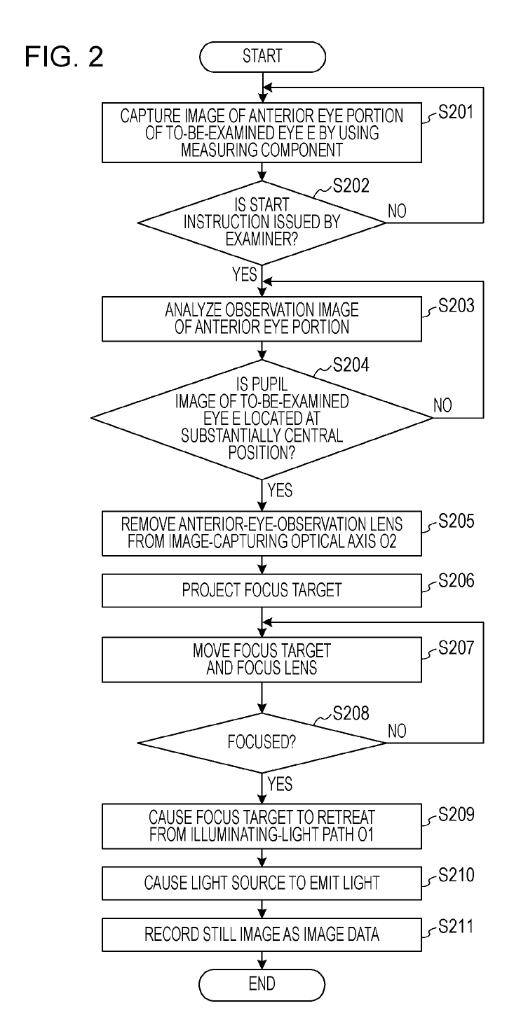

FIG. 2 is a flowchart illustrating a process that is performed by the control unit 13 in the ophthalmic apparatus 100 according to the present embodiment when an image of the to-be-examined eye E is captured.

In step S201, after the ophthalmic apparatus 100 has been switched on by an examiner, the control unit 13 captures an image of an anterior eye portion of the to-be-examined eye E via the image-capturing unit 12. Note that, immediately after the ophthalmic apparatus 100 has been switched on, and the eyepiece portion 11 has been brought into contact with a portion of the face of a patient around the to-be-examined eye E, the anterior-eye-observation lens 220 is placed in line with the image-capturing optical axis O2.

In step S202, the control unit 13 determines whether a start instruction has been issued by the examiner via an operation button 302, which will be described later.

In step S203, the control unit 13 analyzes an observation image of the anterior eye portion.

In step S204, the control unit 13 determines whether a pupil image of the to-be-examined eye E is located at a substantially central position in a video image that is been captured. In the case where the pupil image of the to-be-examined eye E is located at a substantially central position in the video image, the process proceeds to step S205, and in the case where the pupil image of the to-be-examined eye E is not located at a substantially central position in the video image, the process returns to step S203.

In step S205, the control unit 13 removes the anterior-eye-observation lens 220 from the image-capturing optical axis O2.

In step S206, the control unit 13 projects the focus target.

In step S207, the control unit 13 moves the focus target and the focus lens 213.

In step S208, the control unit 13 determines whether the focus lens 213 is focused. In the case where the focus lens 213 is focused, the process proceeds to step S209, and in the case where the focus lens 213 is not focused, the process returns to step S207.

In step S209, the control unit 13 causes the focus target to retreat from the illuminating-light path O1. In step S210, the control unit 13 causes the infrared LED light source 201 or the white LED light source 202 to emit light.

In step S211, the control unit 13 records to the memory or the storage medium as image data a still image of the to-be-examined eye E, which has been undergoing image capturing, and the process is completed.

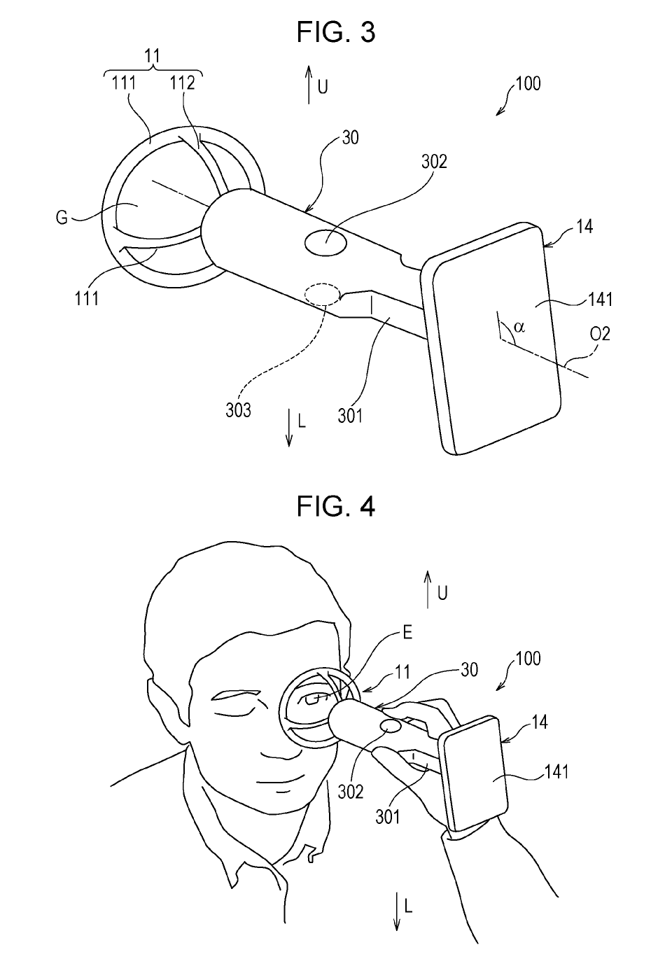

FIG. 3 is a perspective view illustrating the appearance of the ophthalmic apparatus 100 according to the present embodiment.

As illustrated in FIG. 3, the ophthalmic apparatus 100 includes the eyepiece portion 11, a main body 30 in which the image-capturing unit 12 and the control unit 13 are accommodated, and the display 14. In the ophthalmic apparatus 100 according to the present embodiment, the eyepiece portion 11 and the display 14 are positioned on a substantially straight axis in such a manner that the main body 30 is interposed between the eyepiece portion 11 and the display 14, and the ophthalmic apparatus 100 includes a holding portion 301 that is to be hand-held and that is positioned on the straight axis. The configurations of these components will be specifically described below. Note that, in the drawings including FIG. 3, the upper side of the ophthalmic apparatus 100 is represented by the reference letter “U”, and the lower side of the ophthalmic apparatus 100 is represented by the reference letter “L” as necessary.

The eyepiece portion 11 is arranged at a first end of the main body 30 in the axial direction of the main body 30. The eyepiece portion 11 is brought into contact with the face of a patient, specifically, a portion of the face of the patient around the to-be-examined eye E. The portion of the face of the patient around the to-be-examined eye E will hereinafter be referred to as a peripheral portion of the to-be-examined eye E. In the eyepiece portion 11 according to the present embodiment, a contact portion 111 and connection portions 112 are integrally formed. The contact portion 111 is formed in an annular shape larger than the to-be-examined eye E and is brought into contact with the peripheral portion of the to-be-examined eye E. The connection portions 112 connect the main body 30 and the contact portion 111. A gap G is formed between each two of the connection portions 112 that are adjacent to each other. Accordingly, an examiner can bring the contact portion 111 into contact with the peripheral portion of the to-be-examined eye E while visually examining the to-be-examined eye E through the gaps G. Therefore, the probability of deviation occurring in the positions of the ophthalmic apparatus 100 and the to-be-examined eye E can be reduced, and such deviation can be prevented from occurring. Note that each of the connection portions 112 according to the present embodiment may have a shape that covers the to-be-examined eye E with no gap formed between the connecting portion 112 and the adjacent connecting portion 112. In this case, by making the eyepiece portion 11 from a transparent material, the examiner can bring the contact portion 111 into contact with the peripheral portion of the to-be-examined eye E while visually examining the to-be-examined eye E.

The main body 30 connects the eyepiece portion 11 and the display 14 and is formed in a substantially cylindrical shape (rod-like shape or shaft-like shape) having an axis parallel to the image-capturing optical axis O2 of the image-capturing unit 12. The main body 30 includes the holding portion 301 that is to be hand-held and that enables the examiner to hold the ophthalmic apparatus 100 with their hand. The holding portion 301 is formed in a portion of the main body 30 on the side on which the display 14 is disposed, and the holding portion 301 extends in the axial direction of the main body 30. More specifically, the two side surfaces of the holding portion 301 are cut away in the axial direction of the main body 30, so that the holding portion 301 is formed in a constricted (narrow) shape. The examiner can stably hold the ophthalmic apparatus 100 by, for example, directing the index finger tip and the thumb tip toward the eyepiece portion 11 and supporting a portion of the holding portion 301 on the side on which the display 14 is disposed between the base of the index finger and the base of the thumb. Note that the length of the holding portion 301 in the axial direction may be 4 cm or larger from the standpoint of being easily held, and the length of the holding portion 301 in the axial direction may be 8 cm or smaller from the standpoint of preventing an increase in the size of the ophthalmic apparatus 100. Preferably, the length of the holding portion 301 in the axial direction is about 6 cm. Note that the above-mentioned lengths are examples, and the length of the holding portion 301 in the axial direction is not limited to these values and can be a different value.

In addition, in order to hold the ophthalmic apparatus 100 more stably, a protruding portion may be formed at the center of the main body 30. In this case, the protruding portion and a portion of the main body 30 between the protruding portion and the display 14 form the holding portion 301.

The main body 30 includes the operation button 302. Once the examiner presses the operation button 302, the control unit 13 starts performing control. The operation button 302 is arranged in the vicinity of a position where the examiner can press the operation button 302 with their index finger tip when the examiner holds the holding portion 301. More specifically, the operation button 302 is arranged at a substantially central position on the top surface of the main body 30 in the axial direction of the main body 30.

The main body 30 may include two or more operation buttons. The main body 30 according to the present embodiment includes an operation button 303 arranged on the side opposite to the side on which the operation button 302 is arranged. More specifically, the operation button 303 is arranged at a substantially central position on the bottom surface of the main body 30 in the axial direction of the main body 30. Similarly to the operation button 302, once the examiner presses the operation button 303, the control unit 13 starts performing control. As described above, the main body 30 is provided with the operation button 303, so that the examiner can press the operation button 303 with their finger other than their index finger when the examiner holds the main body 30, and accordingly, the operability of the ophthalmic apparatus 100 can be improved.

There is a case where the examiner moves to a position on the side on which the head of a patient is present and performs image capturing when an image of an eye to be examined is captured in a state where the patient is laid down. In this case, the examiner holds the ophthalmic apparatus 100 while the back of their hand is located on the side on which the operation button 302 is present, and thus, it is easier for the examiner to press the operation button 303 than the operation button 302.

Note that, in the present embodiment, although it is assumed that the operation buttons 302 and 303 are constantly effective, the present invention is not limited to this configuration, and one of the operation buttons 302 and 303 may be effective. For example, the operation button 302 may be constantly effective as an operation button for normal use, and the operation button 303 may become effective when an examiner performs a certain operation. For example, the control unit 13 can acquire information, which indicates whether the examiner has performed the certain operation, by using the functionality of the touch panel, which is disposed on the display 14.

The ophthalmic apparatus 100 may include a sensor, such as an acceleration sensor, and the operation button 303 may be controlled to be effective or not to be effective on the basis of information that indicates the position of the ophthalmic apparatus 100 and that is acquired by the sensor. For example, as described above, when an image of an eye to be examined is captured in a state where a patient is laid down, there is a probability that the operation button 303 will be used, and thus, when the sensor detects that the ophthalmic apparatus 100 faces downward, the control unit 13 may control the operation button 303 to be effective. Note that, in the case where the sensor detects that the ophthalmic apparatus 100 faces downward, the control unit 13 may control the display 14 to display a display form that prompts the examiner to choose whether to make the operation button 303 effective. In other words, the operation button 303 will not be immediately effective in response to an output of the sensor, and the examiner is allowed to choose whether to make the operation button 303 effective. Note that the information, which has been acquired by the sensor, is acquired by the control unit 13, and the control unit 13 controls the operation button 303 to be effective or not to be effective on the basis of the acquired information.

As described above, the operation button 303 becomes effective only when necessary, so that image capturing can be prevented from being performed as a result of the operation button 303 being unintentionally pressed by the examiner.

The display 14 is arranged at a second end of the main body 30 in the axial direction of the main body 30. The display 14 is formed in a substantially rectangular plate-like shape. For example, a liquid crystal display can be used as the display 14. The display 14 has a display surface 141. The display surface 141 according to the present embodiment crosses the axis of the main body 30 and is inclined with respect to the axial direction of the main body 30. More specifically, an angle α (obtuse angle) that is formed by the image-capturing-light axis O2 and the display surface 141 and that is illustrated in FIG. 3 is about 120 degrees. In other words, a direction normal to the display surface 141 is a diagonally upward direction. Thus, the examiner can see the display surface 141 from a direction substantially crossing to the display surface 141. Note that the value of the above-mentioned angle α is an example. The present invention is not limited to the value, and the angle α may be a different value.

A display (hereinafter referred to as mobile terminal) such as a smartphone or a tablet terminal may be used as the display 14. In other words, the mobile terminal, which serves as the display 14, may be detachably mounted on the main body 30. In this case, by arranging a to-be-attached portion to which the mobile terminal is to be attached at the second end of the main body 30 in the axial direction of the main body 30, the mobile terminal can easily be mounted on the main body 30, and the mobile terminal can be integrally disposed with the main body 30 like the display 14 integrally disposed with the main body 30. In this case, communication between the control unit 13, which is disposed in the main body 30, and the mobile terminal, such as display control and transmission and reception of image data, can be performed by using a wireless communication unit, such as Bluetooth (Registered Trademark) or Wi-Fi.

FIG. 4 is a diagram illustrating an exemplary usage of the ophthalmic apparatus 100.

As illustrated in FIG. 4, an examiner holds the main body 30 and brings the eyepiece portion 11 into contact with the face of a patient. In this case, the examiner makes a movement similar to that when the examiner holds a pen and presses the pen against a board or the like. In general, such a movement is familiar, and thus, the position and angle of the ophthalmic apparatus 100 with respect to the face of the patient can be stably maintained.

The ophthalmic apparatus 100 includes the operation button 302 that is disposed at a position where the operation button 302 is to be in the vicinity of an examiner's fingertip in the main body 30, which has a rod-like shape and whose axis is parallel to the image-capturing-light axis O2 of the image-capturing unit 12. Thus, the examiner presses the operation button 302 without changing the way in which the examiner holds the ophthalmic apparatus 100, and the control unit 13 starts its operation. In other words, the examiner can easily press the operation button 302, and thus, the position and angle of the ophthalmic apparatus 100 with respect to the face of a patient can be stably maintained.

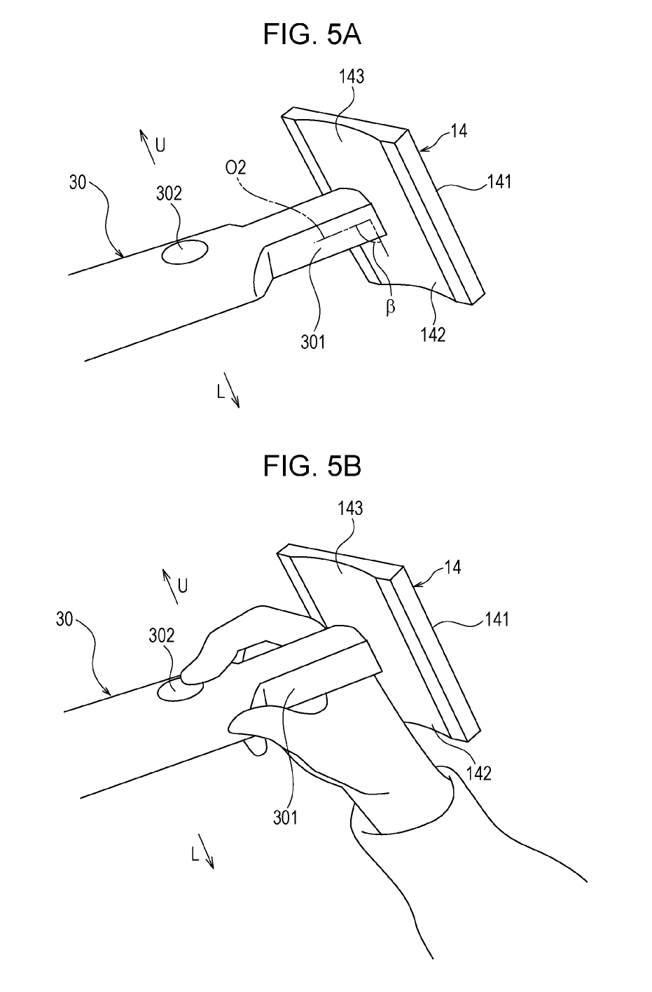

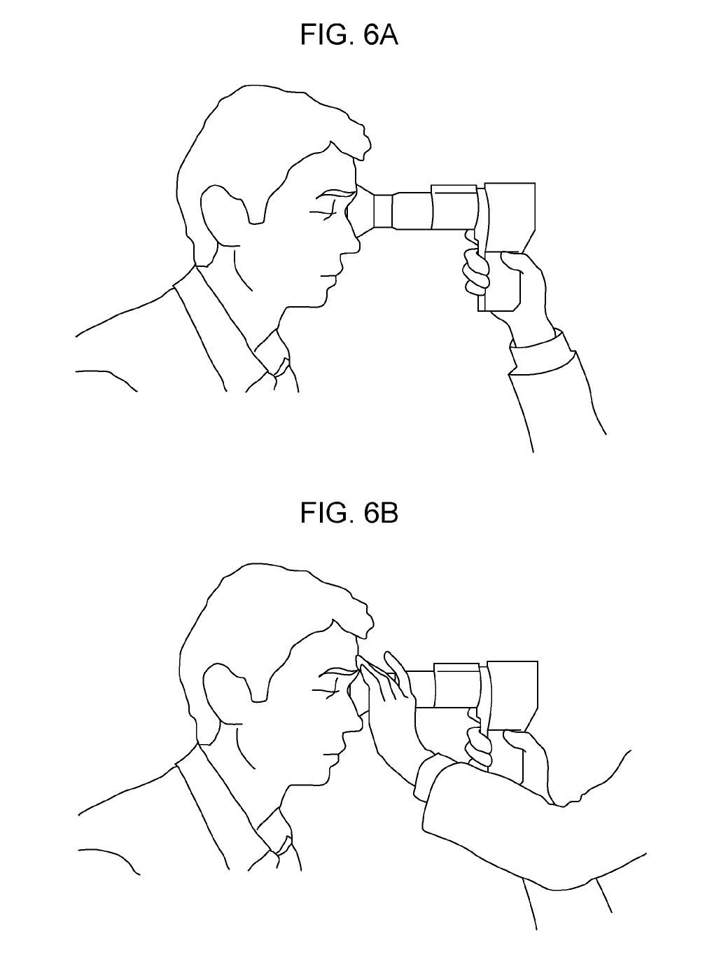

FIG. 5A is a perspective view illustrating the display 14 of the ophthalmic apparatus 100 as seen from the side opposite to the display surface 141. FIG. 5B is a perspective view illustrating a state where an examiner is holding the holding portion 301.

As illustrated in FIG. 5B, in a state where the examiner is holding the holding portion 301, the back of the examiner's hand is in contact with a rear surface 142 of the display 14. As a result of the back of the examiner's hand is brought into contact with the rear surface 142, the contact area between the examiner's hand and the ophthalmic apparatus 100 increases, and thus, the position and angle of the ophthalmic apparatus 100 with respect to the face of a patient can be more stably maintained.

The rear surface 142 according to the present embodiment is inclined with respect to the axial direction of the main body 30. More specifically, an angle β (obtuse angle) that is formed by the image-capturing-light axis O2 and the rear surface 142 and that is illustrated in FIG. 5A is about 120 degrees. In other words, a direction normal to the rear surface 142 is a diagonally downward direction. Since the direction in which the rear surface 142 is inclined and the direction in which the back of the examiner's hand is inclined when the examiner is holding the holding portion 301 match each other, the contact area between the back of the examiner's hand and the rear surface 142 can be large. Note that the value of the above-mentioned angle β is an example. The present invention is not limited to the value, and the angle β may be a different value.

The rear surface 142 according to the present embodiment includes a curved portion 143 that is curved in a recessed manner and that extends over the entire length of the display 14 in a top-bottom direction such that the back of a hand may be more easily brought into contact to the rear surface 142. Note that a probability of the back of a hand sliding over the rear surface 142 can be reduced by forming a layer made of a flexible material on the rear surface 142. Note that the rear surface 142 is not limited to be formed in a recessed manner and may be formed in a different shape. For example, the rear surface 142 may be formed in a convex shape in such a manner as to increase the contact area between the rear surface 142 and a portion between the base of the thumb and the base of the index finger. For example, the curved portion 143, which is formed in a recessed manner, can be replaced with a curved portion having a convex shape.

Although the display surface 141 and the rear surface 142 are disposed in such a manner as to be parallel to each other in the present embodiment, the present invention is not limited to this configuration, and the display surface 141 and the rear surface 142 need not be parallel to each other. In other words, the angle α formed by the image-capturing-light axis O2 and the display surface 141 and the angle β formed by the image-capturing-light axis O2 and the rear surface 142 may be different from each other. For example, the angle α formed by the image-capturing-light axis O2 and the display surface 141 may be 90 degrees. Note that the angle α formed by the image-capturing-light axis O2 and the display surface 141 is not limited to this value and may be a different value. In the case where the angle α formed by the image-capturing-light axis O2 and the display surface 141 is larger than 90 degrees, an examiner can easily check the display surface 141 in a state where the position of an eye to be examined is lower than the positions of the eyes of the examiner. In the case where the angle α formed by the image-capturing-light axis O2 and the display surface 141 is 90 degrees, the examiner can easily check the display surface 141 when the position of the eye to be examined and the positions of the eyes of the examiner are at substantially the same level. In addition, the examiner can examine the eye to be examined and check the display surface 141 at the same time, and thus, positional adjustment of the ophthalmic apparatus 100 and the like can be facilitated.

As described above, according to the present embodiment, when an image of an eye to be examined is captured while the ophthalmic apparatus 100 is hand-held by an examiner, the position and angle of the ophthalmic apparatus 100 with respect to the face of a patient can be stably maintained.

Since the display surface 141 of the display 14 crosses the axial direction of the main body 30, the examiner can easily see the display surface 141.

Since, in the main body 30, the holding portion 301 is formed on the side on which the display 14 is disposed, and the rear surface 142 of the display 14, which is on the opposite side to the display surface 141, makes contact with the back of a hand that is holding the holding portion 301, the position and angle of the ophthalmic apparatus 100 with respect to the face of a patient can be more stably maintained.

Since the rear surface 142 is inclined with respect to the axial direction of the main body 30, the contact area between the back of the hand of the examiner and the rear surface 142 can be large.

Since a layer having flexibility is formed on the rear surface 142, the probability of the back of the hand sliding over the rear surface 142 can be reduced.

Since the eyepiece portion 11 is formed in such a manner that the examiner can visually examine the to-be-examined eye E when the eyepiece portion 11 is in contact with the to-be-examined eye E, deviation in the positions of the ophthalmic apparatus 100 and the to-be-examined eye E can be prevented from occurring or the probability of such deviation occurring can be reduced.

Since the main body 30 includes the two operation buttons 302 and 303, one of the operation buttons 302 and 303 can be pressed by a finger other than a particular finger, and the operability of the ophthalmic apparatus 100 can be improved.

The display 14 may be detachably mounted on the main body 30, and in this case, a smartphone or a tablet terminal can be used as the display 14. In addition, in this case, by arranging the to-be-attached portion, to which the display 14 is to be attached, at the second end of the main body 30 in the axial direction of the main body 30, the display 14 can easily be mounted on the main body 30.

Although the embodiment of the present invention has been described above, the present invention is not limited to the above-described embodiment, and modifications can be made within the scope of the present invention.

For example, although the case where the eyepiece portion 11, which has been described above, includes the contact portion 111 having an annular shape, and the contact portion 111 is brought into contact with the peripheral portion of the to-be-examined eye E has been described, the present invention is not limited to this case. For example, the contact portion 111 does not need to have an annular shape and may have a shape with which the contact portion 111 comes into contact with at least a portion of the peripheral portion of the to-be-examined eye E or a shape with which the contact portion 111 comes into contact with the peripheral portion of the to-be-examined eye E at two or more points.

In addition, although the case where both the above-described operation buttons 302 and 303 are buttons that are used for issuing an instruction to the control unit 13 to start a control operation has been described, the operation buttons 302 and 303 may be buttons each of which is used for issuing a different control instruction.

The present invention may be achieved by performing the following process. That is to say, in the process, programs for realizing the functions described in the above embodiment are supplied to the ophthalmic apparatus 100 via a network, various computer readable storage media, or the like, and the control unit 13 (computer) of the ophthalmic apparatus 100 reads and executes the programs.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-112662, filed May 30, 2014, which is hereby incorporated by reference herein in its entirety.

What is claimed is:

1. An ophthalmic apparatus comprising:
an image-capturing unit that captures an image of an eye to be examined;
a main body having a rod-like shape and that accommodates the image-capturing unit;
an eyepiece portion that is positioned at a first end of the main body in an axial direction of the main body and that is brought into contact with a portion around the eye to be examined; and
a display that is positioned at a second end of the main body in the axial direction of the main body and that displays an image of the eye to be examined,
wherein the main body includes a holding portion that is to be held by a hand of an examiner and that extends in the axial direction of the main body.

2. The ophthalmic apparatus according to claim 1,
wherein a display surface of the display crosses the axial direction of the main body.

3. The ophthalmic apparatus according to claim 1,
wherein the display surface of the display is inclined with respect to the axial direction of the main body.

4. The ophthalmic apparatus according to claim 1,
wherein, in the main body, the holding portion is formed on a side on which the display is disposed, and
wherein a rear surface of the display, which is on an opposite side to the display surface, is brought into contact with the back of the hand that holds the holding portion.

5. The ophthalmic apparatus according to claim 4,
wherein the rear surface is inclined with respect to the axial direction of the main body.

6. The ophthalmic apparatus according to claim 4,
wherein a layer having flexibility is formed on the rear surface.

7. The ophthalmic apparatus according to claim 4,
wherein the rear surface is formed in a convex manner or in a recessed manner.

8. The ophthalmic apparatus according to claim 1,
wherein the eyepiece portion enables the examiner to visually examine the eye to be examined when the eyepiece portion is in contact with the portion around the eye to be examined.

9. The ophthalmic apparatus according to claim 1,
wherein the main body includes two or more operation buttons.

10. The ophthalmic apparatus according to claim 1,
wherein the display is detachably mounted on the main body.

11. The ophthalmic apparatus according to claim 10,
wherein the main body includes a to-be-attached portion, to which the display is to be attached, at the second end of the main body in the axial direction of the main body.
